(19) United States
(12) Patent Application Publication
(10) Pub. No.: US 2022/0192517 A1
(43) Pub. Date: Jun. 23, 2022

(54) SYSTEMS AND METHODS FOR DETECTION OF PLAQUE AND VESSEL CONSTRICTION

(71) Applicant: Dyad Medical, Inc., Allston, MA (US)

(72) Inventors: David A. Vader, Concord, MA (US); Ronny SHALEV, Brookline, MA (US); John LONG, Shrewsbury, MA (US)

(21) Appl. No.: 17/132,654

(22) Filed: Dec. 23, 2020

(51) Int. Cl. A61B 5/02; A61B 5/00; G06T 7/00; G06T 7/11; G06T 7/168; G06T 3/40

(52) U.S. Cl. CPC A61B 5/02007; A61B 5/0066; A61B 5/0084; G06T 7/0012; G06T 2207/30101; G06T 7/168; G06T 3/40; G06T 2207/10101; G06T 2207/20056; G06T 7/11

(57) ABSTRACT

A method and system for detecting plaque and vessel constriction by processing intracoronary optical coherence tomography (IVOCT) pullback data performed by software executed on a computer. One example method includes inputting IVOCT pullback data from an imaging device, performing full semantic segmentation of the every image of the IVOCT pullback data with a frame-based segmentation module, generating a cross-sectional frame-based image of the every image of the segmented IVOCT pullback data with a cross-sectional display, and determining the presence of plaque and vessel constriction with an automated analysis application analyzing the cross-sectional frame-based images.

### TECHNICAL FIELD

This specification describes examples of plaque and vessel constriction detection and analysis using intravascular optical Coherence Tomography (IVOCT).

### BACKGROUND

Coronary artery disease (CAD) is one of the most common forms of heart disease, which is the leading cause of death in developed countries. Early detection and accurate assessment of CAD is crucial in the identification of patients at risk of these highly common yet usually preventable coronary events. In CAD, the chief culprit is usually the build-up of plaque, specifically soft-plaque, in the arteries. Accordingly, there is high interest in the medical community in detecting coronary artery disease.

Optical Coherence Tomography (OCT) is commonly used in intravascular imaging for the detection of plaque and the constriction of blood vessels. OCT has also been utilized for imaging stents and for evaluating the quality of their apposition after therapeutic placement to address latter issues. Earlier detection of plaque, as well as distinction between the different types of plaque, is desirable to maximize treatment options and outcome.

At present, detection of plaque is generally done manually by the physician acquiring and reviewing the image pullbacks obtained from intravascular optical coherence tomography (IVOCT). Given the large number of cross-sectional frames to review and difficulty of identifying plaque by pure visual inspections, this presents a challenge in terms of the skill and time required to perform a proper assessment of the image data.

There exists a need for a computerized and automated plaque detection solution that can address these problems by reducing time and labor costs and by increasing reliability and reproducibility of stent analysis results. The availability of a significant annotated dataset of IVOCT pullbacks, coupled with recent advances in deep learning-based image segmentation provide a ripe opportunity to train an algorithm for real-time automated detection of plaque in cross-sectional views. Moreover, succinct and effective communication of these results at a scan-level for further manual investigation is an important component of a commercial solution.

### SUMMARY

In one embodiment, a method for detecting plaque and vessel constriction by processing intracoronary optical coherence tomography (IVOCT) pullback data performed by software executed on a computer is provided. The method includes inputting IVOCT pullback data from an imaging device, performing full semantic segmentation of the every image of the IVOCT pullback data with a frame-based segmentation module, generating a cross-sectional frame-based image of the every image of the segmented IVOCT pullback data with a cross-sectional display, and determining the presence of plaque and vessel constriction with an automated analysis application analyzing the cross-sectional frame-based images.

In another embodiment, a system for detecting plaque and vessel constriction by processing intracoronary optical coherence tomography (IVOCT) pullback data performed by software executed on a computer is provided. The system includes an IVOCT device for acquiring IVOCT pullback data from a patient, a computer for processing the IVOCT pullback data with a method for detecting plaque and vessel constriction where the method includes inputting IVOCT pullback data from an imaging device, performing full semantic segmentation of the every image of the IVOCT pullback data with a frame-based segmentation module, generating a cross-sectional frame-based image of the every image of the segmented IVOCT pullback data with a cross-sectional display, and determining the presence of plaque and vessel constriction with an automated analysis application analyzing the cross-sectional frame-based images, and a display screen to display the IVOCT pullback data and the cross-sectional frame-based generated by the method.

### DESCRIPTION OF THE DRAWINGS

The accompanying drawings, which are incorporated in and constitute a part of the specification, illustrate various example methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. Furthermore, elements may not be drawn to scale.

FIG. 1A illustrates an exemplary flowchart of an example method for visualization and analysis of IVOCT image pullbacks for plaque detection in accordance with one illustrative embodiment.

FIG. 1B illustrates an exemplary schematic of the example method for visualization and analysis of IVOCT image pullbacks for plaque detection in accordance with FIG. 1A.

FIG. 2 illustrates an exemplary flowchart of frame-based pre-processing module of the method for visualization and analysis of IVOCT image pullbacks for plaque detection in accordance with FIG. 1A.

FIG. 3A illustrates an exemplary flowchart depicting the modified software architecture of the frame-based feature segmentation module in accordance with FIG. 1A.

FIG. 3B illustrates an exemplary flowchart of the contracting block of the modified U-Net software architecture in accordance with FIG. 3A.

FIG. 3C illustrates an exemplary flowchart of the expanding block of the modified U-Net software architecture in accordance with FIG. 3A.

FIG. 3D illustrates an exemplary flowchart of concatenation depicted in U-Net software architecture in accordance with FIG. 3A.

FIG. 4 illustrates an exemplary flowchart of the evaluation, data generation, and image generation of method for visualization and analysis of IVOCT image pullbacks for plaque detection in accordance with FIG. 1A

FIGS. 5A-5C illustrate exemplary graphs of pixel-based receiver operating characteristics (ROC) curves of each class of plaque in accordance with the method for visualization and analysis of IVOCT image pullbacks for plaque detection in accordance with FIG. 1A

FIG. 6 presents exemplary IVOCT cross-sectional image outputs by the frame-based cross-sectional display module in accordance with FIG. 1A.

FIG. 7A presents exemplary raw Enface image outputs by the Enface display module in accordance with FIG. 1A.

FIG. 7B presents exemplary Enface plaque overlay image outputs by the Enface display module in accordance with FIG. 1A.

FIG. 7C presents exemplary mixed Enface image outputs by the Enface display module in accordance with FIG. 1A.

### DETAILED DESCRIPTION

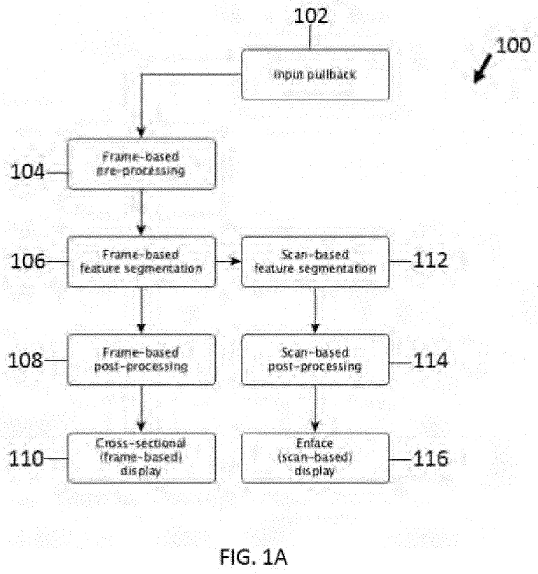

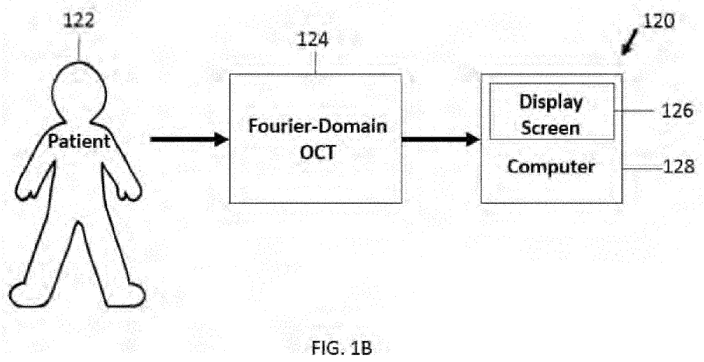

FIGS. 1A and 1B illustrate an exemplary method 100 and system 130 visualization and analysis of IVOCT image pullbacks for plaque detection in accordance with one illustrative embodiment. The method 100 depicts the overview of an algorithm which automatically detects the plaque from IVOCT image pullbacks. The method 100 also determines the type of plaque distinctly from each other. In one embodiment, the automated algorithm of the computerized method 100 of FIG. 1A utilizes the IVOCT input pullback 102 real clinical examinations following which the data from the IVOCT input pullback 102 is subject to a frame-based pre-processing module 104 in which the data is converted for analysis. The converted data is segmented by a frame-based feature segmentation module 106 following which the output of the frame-based feature segmentation module 106 is subject to post-processing by a frame-based post processing module 108 before presenting IVOCT cross-sectional images via a cross-sectional frame-based display 110. In another embodiment, the automated algorithm of the computerized method 100 of FIG. 1A, in addition to the frame-based feature segmentation, also carries out scan-based feature segmentation utilizing the output of the frame-based feature segmentation module 106 and processing the output with a scan-based feature segmentation module 112. The output of the scan-based feature segmentation module 112 is further subject to post-processing by a scan-based post processing module 114 before presenting a scan based display of the IVOCT images via an Enface scan-based display 116.

The method 100 can be performed by a computer 128 connected to a Fourier-Domain OCT system 124 used to acquire IVOCT image pullbacks from a patient 122 as shown in FIG. 1B. The computer includes a display screen 126 to display the pullbacks, as well as a plaque segmentation computed and displayed by the method 100. Preferably, the method 100 will detect the presence and the type of plaque for evaluation and follow-ups for display to a clinician for further manual investigation. In one embodiment, the automated algorithm of the computerized method 100 of FIG. 1A utilizes the IVOCT input pullback 102 from real clinical exams conducted with the Fourier-Domain OCT system 124 on patients 122. Each clinical examination yields data for the input pullback 102, which is a collection of images analyzing a section of a blood vessel. In one embodiment, the Fourier-Domain OCT system 124 was equipped with a tunable laser light source sweeping from 1250 nm to 1370 nm, providing 15 μm resolution along an A-line. The input pullback 102 from the Fourier-Domain OCT 134 was acquired at speed of 20 mm/sec over a distance of 54.2 mm with a 200 μm interval between frames, giving 217-375 total images (also referred to as frames). These frames were collected in the r-theta format. Each polar-coordinate (r, θ) image consisted of at least 504 A-lines and at least 900 pixels along the A-line, and 16 bits of gray-scale data.

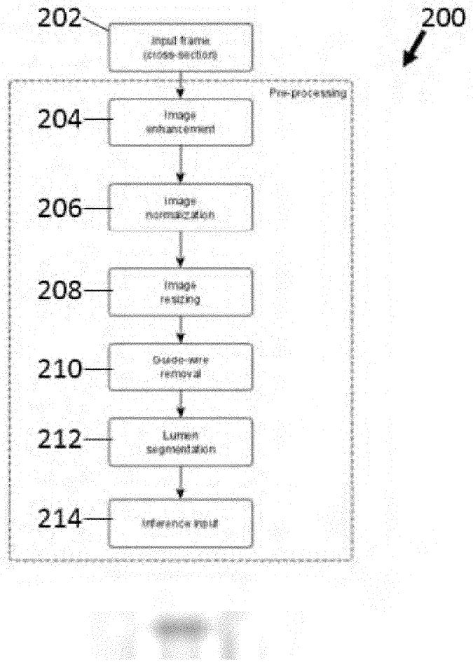

FIG. 2 illustrates a flowchart 200 of frame-based pre-processing module 104 of the method 100 for visualization and analysis of IVOCT image pullbacks for plaque detection in accordance with FIG. 1A. The IVOCT image pullbacks are used as input 202 of the frame-based pre-processing module 104. In the embodiment described above, the input 202 of the frame-based pre-processing module 104 was utilized from a dataset of 13,936 semantically labeled IVOCT cross-sectional frames extracted from the 179 IVOCT pullbacks. Each IVOCT pullback comprised of 440 cross-sectional frames on average and an average of 71 frames were labeled by an expert clinician with IVOCT experience to annotate three types of plaque (calcium, fibrous, and lipidic). The data from IVOCT pullback images of the input 202 are first subject to data augmentation 203. As part of data augmentation 203, the data from IVOCT pullback images of the input 202 are flipped along the angular axis, which is equivalent to flipping clockwise rotation of the OCT probe into the counter-clockwise direction, or vice versa. Following this, the data is shifted in the axial (radial) direction by up to 20% of the length of the A-line. This is more or less equivalent to increasing or decreasing the diameter of the blood vessel. The data from IVOCT pullback images of the input 202 has uniform noise randomly added, whose maximum amplitude is up to 10% of the image range (255). As a last step of data augmentation, the data from IVOCT pullback images of the input 202 is subject to randomly generated non-uniform rotational distortion (NURD), which simulates deviations from a uniformly rotating OCT probe. The amplitude of simulated NURD is up to 30% of the baseline rotation of the OCT probe and is assumed to be periodic in nature, with a center frequency at 2 times the nominal rotation period of the OCT probe.

The frame-based pre-processing module 104 as shown in FIG. 2 further includes image enhancement 204, image normalization 206, and image resizing 208 modules in which the IVOCT pullback images of the input 202 are organized into folders for each IVOCT pullback image, with each set of manual annotation files (in x-y space) located in the same folder as the raw data. The image enhancement 204, image normalization 206, and image resizing 208 modules are then utilized to read the manual annotations of the IVOCT images for conversion to R-theta space, at the corresponding resolution of the raw OCT data. The combined R-theta annotations and the labels extracted from previously-trained neural networks for each frame of the IVOCT images are used to generate consistent pixel-level annotations for each cross-sectional frame of every IVOCT pullback image. Following the latter, the data from the IVOCT pullback images of the input 202 are resized and resampled to the correct input resolution needed by the frame-based feature segmentation module 106. The frame-based pre-processing module 104 as shown in FIG. 2 also includes a guide-wire removal module 210 module and a lumen segmentation module 212 to process the data from the IVOCT pullback images of the input 202 to detect guide wires, struts, the inner lumen, and also to classify other pixels with significant signal-to-noise-ratio not determined to be guide wires, struts, inner lumen, or plaque with the tissue label. The data from the IVOCT pullback images of the input 202 is processed by all the modules of the frame-based pre-processing module 104 to prepare an inference input 214 comprising pre-processed two dimensional (2D) log-scale data compatible with input for the frame-based feature segmentation module 106.

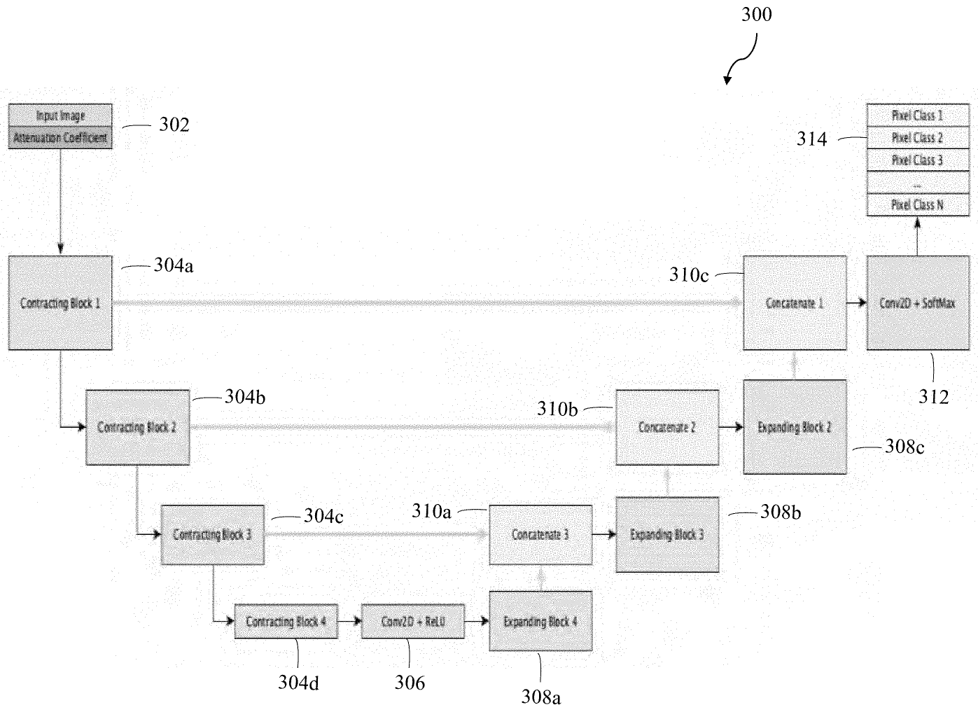

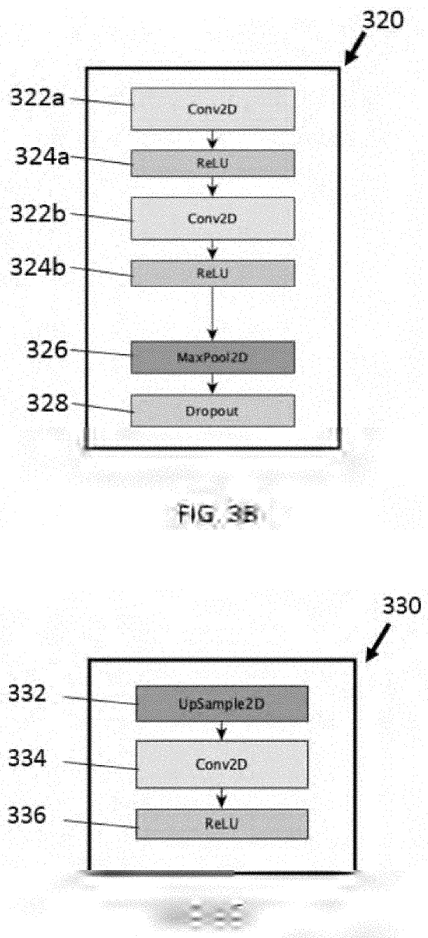

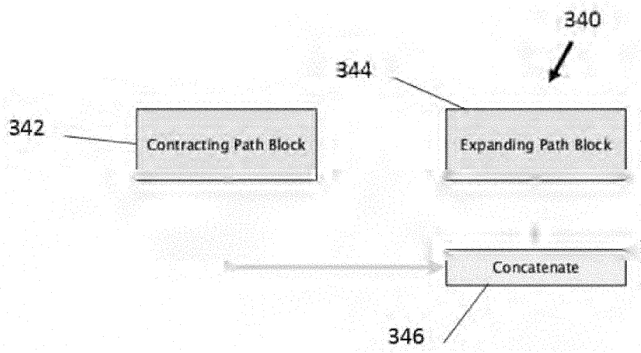

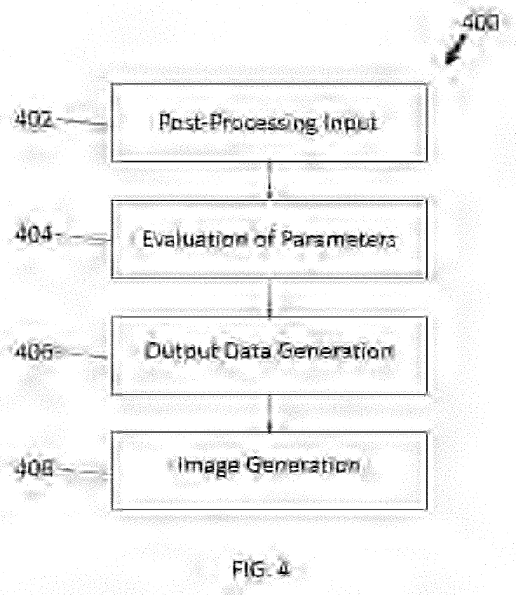

FIG. 3A illustrates an example flowchart 300 depicting the modified U-Net software architecture of the frame-based feature segmentation module 106 in accordance with FIG. 1A. The frame-based segmentation module 106 is used to process the inference input 214 generated by the pre-processing module 104 and was created with a modified variation of a software architecture based on U-Net architecture for semantic segmentation. In one embodiment, the software architecture of the frame-based segmentation module 106 includes an input block 302 for accepting the inference input 214 generated by the pre-processing module 104, contracting blocks 304a-d, a convolution block with a relu activation function 306, expanding blocks 308a-c, concatenating blocks 310a-c, a final convolution block with a SoftMax activation function 306, and an output block 314. The input block 302 processes the inference input 214 to compute and append one additional image channel, which is the attenuation coefficient. The inference input 214 is a single, and gray-scale channel image with pixel values between 0 and 255. In one embodiment of the present disclosure, the attenuation coefficient of inference input 214 is gamma-adjusted and scaled to fit into the 0-255 pixel value range and both resulting channels output from block 302 are normalized as indicated earlier, by subtracting 127.5 and dividing by 127.5, to force the pixel values into the [−1,1] range. In the same embodiment described above, the frame-based segmentation module 106 is adjusted to utilize an Adam optimizer, have a learning rate scheduler to multiply the learning rate of the frame-based segmentation module 106 by a chosen factor of 0.8 every 10 epochs for a total of 50 epochs, use a batch size of 4 images, have 3 augmentations loaded per sample in each epoch, use 3-fold cross-validation for performance evaluations, utilize label smoothing in ground truth labels to account for potential errors in labeling, and save model weights every 5 epochs.

FIGS. 3B and 3C illustrates exemplary flowcharts 320 and 330 of the contracting block 304 and the expanding block 308 of the modified U-Net software architecture in accordance with FIG. 3A respectively. In one embodiment of the present disclosure, the contracting block 304 includes two 2D convolutional layers 322a-b, two activation functions 324a-b, a 2D maxpooling layer 326, and a dropout layer 328. The convolution layer 322 has a kernel value of 3, and stride value of 1. The 2D maxpooling layer has a kernel value of 3 and stride value of 2. The dropout layer has a probability of 0.2. In one embodiment of the present disclosure, the expanding block 308 includes a 2D upsampling layer 332 with stride set to match the corresponding 2D maxpooling layer 326 in the contracting block 304, a 2D convolutional layer 334 with a kernel value of 3 and with a number of features to match the corresponding contracting path block 304 with a stride value of 1, and a relu activation function block 336. The parameters of the contracting path block 342 are designed to allow concatenation with the parameters of the expanding path blocks 344 as shown in FIG. 3D which presents an example flowchart 340 of concatenation depicted in U-Net software architecture in accordance with FIG. 3A.

FIG. 4 illustrates an exemplary flowchart 400 of the evaluation, data generation, and image generation of method 100 for visualization and analysis of IVOCT image pullbacks for plaque detection in accordance with FIG. 1A. Exemplary flowchart 400 includes evaluation, data generation, and image generation of the frame-based post processing module 108, and the frame-based cross-section display 110 as well as the evaluation, data generation, and image generation of the scan-based feature segmentation 112, scan-based post-processing module 114, and the scan-based Enface display 116. In the post-processing input module 402, the output data of the frame-based processing module 106 is assessed by the frame-based post-processing module 108, the scan-based feature segmentation 112, and the scan-based post processing module 114. The output of the post-processing input module 402 is evaluated by the parameter evaluation module 404. The parameters evaluated by the parameter evaluation module 404 include are listed in Table 1.

TABLE 1

| Parameters | Parameter Significance |
|---|---|
| Categorical Cross- | Weighing the losses for different classes of |
| Entropy Loss | plaque |
| Pixel-level Accuracy | Comparing training and validation |
| | performance |
| Sensitivity (Se) and | Computing receiver-operator characteristic |
| Specificity (Sp) | (ROC) curve and area under the curve (AUC) |
| Youden Index (J), J = | Calculate Jₘₐₓ (maximum threshold) and Jₒₚₜ |
| Se + Sp − 1 | (optimum threshold) |
| Jₘₐₓ/2 | Sensitivity of the quality of the results to the |
| | choice of the threshold |
| 3 × 3 Plaque Confusion | Compute quality of the pixel-level |
| Matrix | segmentation and to track the sum of the 3 |
| | diagonal elements of the confusion matrix |
| | during algorithm training. |
| 2D Dice Overlap | Measuring the per-frame Dice overlap |
| Coefficient (per frame | coefficient between the manually labeled |
| and plaque class) | pixels of that class and the algorithm-detected |
| | pixels of that class |
| 2D Dice Overlap | Measure the per-object Dice overlap |
| Coefficient (per object | coefficient between the manually labeled |
| and plaque class) | pixels of that class and the algorithm-detected |
| | pixels of that class. |
| 1D Dice Overlap | Measure the angular overlap to measure the |
| Coefficient | angular extent of the plaque |
| 1D Angular Ground | Extract traditional binary classifier metrics |
| Truth | such as accuracy, sensitivity and specificity for |
| | each 360-degree cross-sectional frame. |

In one embodiment of the present disclosure, the computation of a number of metrics by the parameter evaluation module 404 is evaluated with cross-sectional data as input for the post-processing input module 402, where the presence or absence of a specific object (plaque) is binarized. To binarize the continuous inference result for each plaque type, thresholding is combined with the application of simple morphological operators (binary opening and closing). The threshold for each of the three classes are chosen to be close to the optimal threshold as evaluated by the evaluation parameter module 404. In the same embodiment described above, the modified U-Net software architecture of the frame-based feature segmentation module 106 is adjusted to optimize per-A-line sensitivity and specificity as evaluated on the validation data; in general, the resulting threshold was higher than the optimal threshold computed on a per-pixel basis, as defined earlier. The kernel sizes for the morphological operators are small (3-7 pixels across) and are primarily intended to eliminate small segmented structures or holes that are assumed to be clinically less relevant.

Results

In the exemplary flowchart 400 depicted in FIG. 4, following the post-processing input module 402 and the parameter evaluation module 404, the model 100 is optimized and the output generated by the optimized method 100 is collated and sorted by the data generation module 406. The data collated by the data generation module 406 is sorted according to the various parameters of study listed in Table 1 as part of the parameter evaluation module 404.

Architecture and Parameters

In the same embodiment of the current disclosure discussed above, the optimal architecture for the frame-based segmentation module 106 is determined to be with the usage of 4 contracting blocks 304, each constituted of two 3×3 kernel convolution blocks 322 and ReLU activation functions 324 followed by a max-pool 326 and a dropout layer 328. Due to the higher resolution of 2D images in the axial direction compared to the angular direction, a max-pool stride of 1 for the first contracting block 304 in the angular direction. Therefore, even though the initial image A-line length is twice as long as the number of A-lines, a square image with equal height and width obtained after the first contracting block 304.

In the same embodiment of the current disclosure discussed above, 16 was determined as the default number of filters of 16 the first convolutional block 304, and the number of filters was doubled for each subsequent convolutional block 304 resulting in a relatively light-weight neural network, with approximately 800,000 trained parameters only.

In the same embodiment of the current disclosure as discussed above, the overall performance was determined to be optimal when all 3 plaques (calcium, fibrous and lipidic) had equally-weighted losses, which correlates with approximately equal pixel-level counts for the ground truth annotation of these 3 classes, as shown in Table 2.

TABLE 2

| | Ground truth labeled pixels (at |
|---|---|
| Plaque type | down-sampled resolution 512 × 256) |
| Calcium | 21,717,710 |
| Fibrous | 17,342,891 |
| Lipidic | 17,241,233 |

Pixel-Level Accuracy and Per-Class AUC

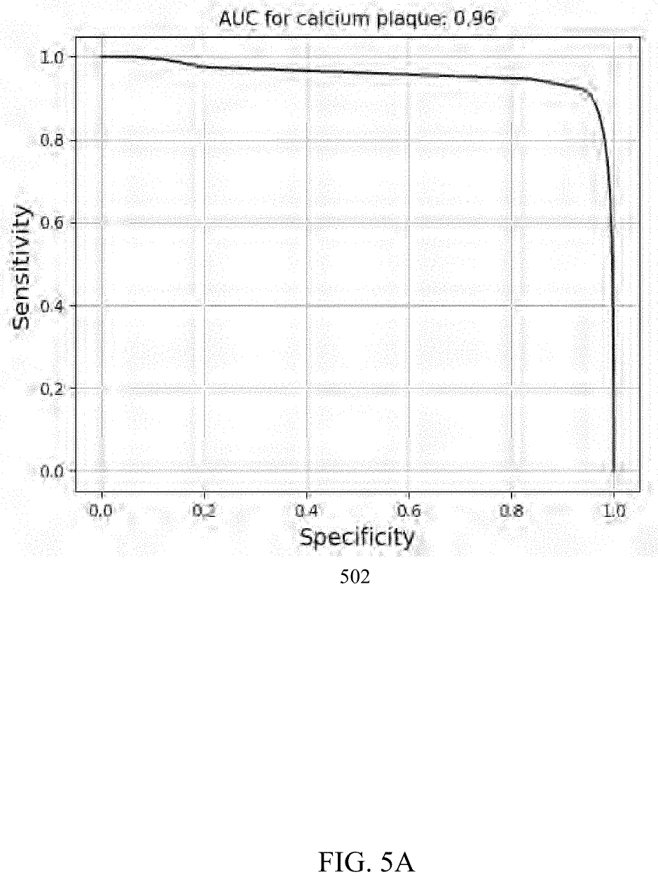

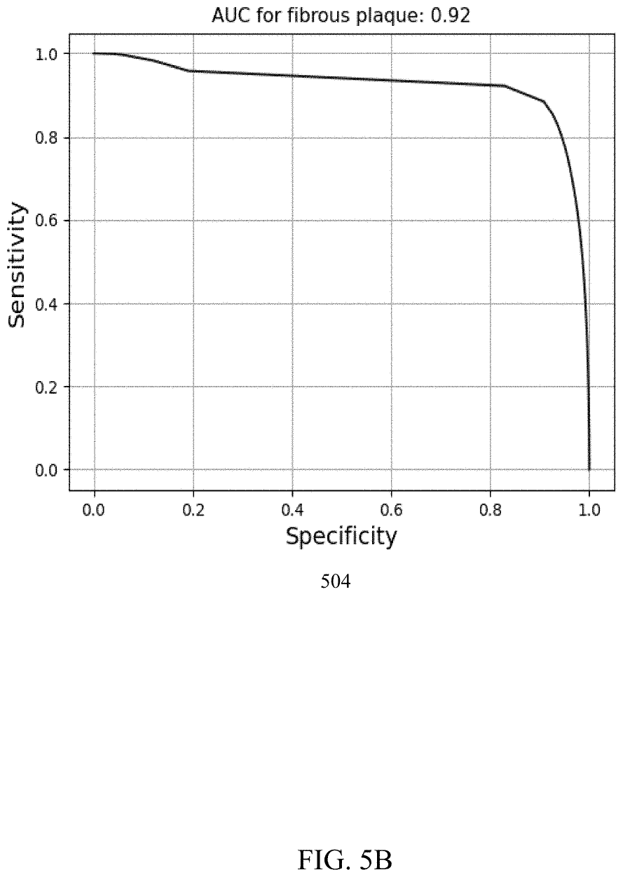

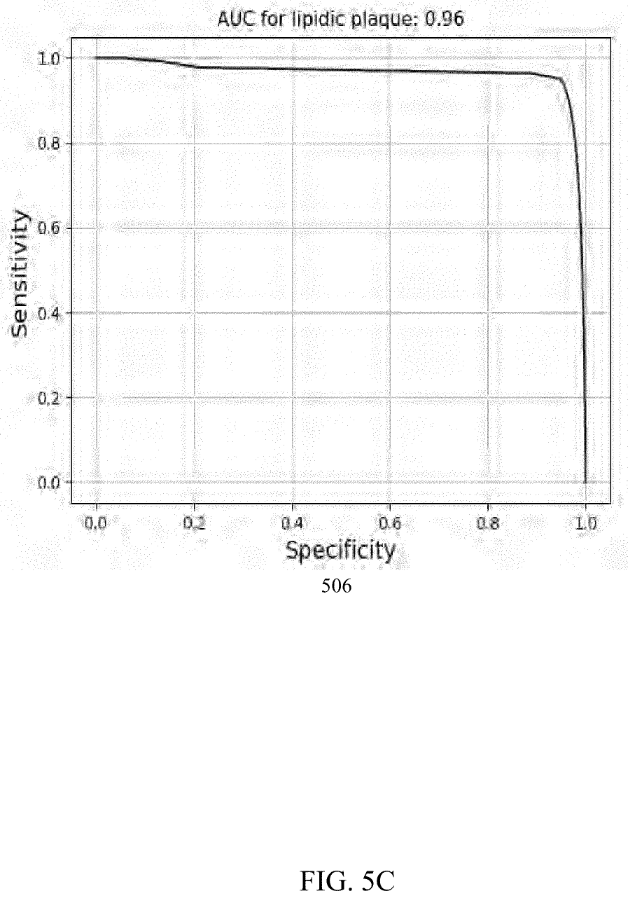

In the above discussed embodiment of the present disclosure, when all classes were combined, the pixel-level accuracy on the training set ranges between 90% and 95%. Validation accuracy is generally in the low 90s, with 90-91% being a typical value during cross-validation. FIGS. 5A-5C illustrate exemplary graphs of pixel-based receiver operating characteristics (ROC) curves of each class of plaque (calcium 502, fibrous 504, and lipidic 506) in accordance with the method 100 for visualization and analysis of IVOCT image pullbacks for plaque detection in accordance with FIG. 1A. The per-class area under the curve (AUC) at the end of each epoch for each class of interest was characterized (including the 3 types of plaque) and cross-validated as presented in Table 3.

TABLE 3

| Class | AUC range |
|---|---|
| Class 0: background | >95% |
| Class 1: general tissue | ~95% |
| Class 2: lumen boundary | ~99% |
| Class 3: lumen | ~99% |
| Class 4: calcium plaque | 90-95% |
| Class 5: calcium plaque | 90-95% |
| Class 6: calcium plaque | 90-95% |
| Class 7: EEM | N/A (not enough training pixels) |
| Class 8: guide wire | ~99% |
| Class 9: guide-wire shadow | >95% |

Optimal Threshold and Range of Robust Thresholds

In the same embodiment of the current disclosure discussed above, the specificity (Sp) and the sensitivity (Se) curves were computed to predict a near optimal threshold within a range 0.08-0.10, which ensures both sensitivity and specificity are balanced when trained with a classifier with 10 target classes. It was determined that the one notable exception is the background class, with an optimal threshold in the range 0.15-0.20.

Plaque Confusion Matrix

In the same embodiment of the current disclosure discussed above, the best confusion matrix for the plaque classes was determined to have a sum of its 3-diagonal elements at approximately 2.2. A typical set of values for the confusion matrix after optimization are:

$$C = \begin{matrix}0.6 & 0.2 & 0.2 \\ 0.1 & 0.8 & 0.1 \\ 0.1 & 0.1 & 0.8\end{matrix}$$

Where the first row corresponds to calcium plaque, second to fibrous plaque and the third to lipidic plaque. It was noted that ground truth for fibrous and lipidic plaque is more readily recognized by the method 100 than ground truth associated with calcium plaque.

Dice Measures of Overlap

In the same embodiment of the current disclosure discussed above, the Dice coefficients were measured for the 3 plaque classes. Both 2D per-object Dice coefficient, which looks at the overlap of pixels in an object of a frame for a given class while treating all pixels as belonging to the same object if they are direct neighbors and the 1D Dice coefficient, which looks at the angular overlap of the ground truth and inference, were calculated and are presented in Table 4. The 2D Dice was measured in x-y space, whereas the angular 1D Dice looks at the R-theta data, specifically at the angular coordinates of labeled plaque.

TABLE 4

| Class | 2D per-object | 1D angular |
|---|---|---|
| Calcium plaque | 0.56 | 0.78 |
| Fibrous plaque | 0.37 | 0.66 |
| Lipidic plaque | 0.60 | 0.80 |

1D Accuracy, Sensitivity and Specificity

In the same embodiment of the current disclosure discussed above, values of the accuracy, sensitivity, and specificity of the 1D Dice coefficient values were tabulated in Table 5.

TABLE 5

| Class | 1D accuracy | 1D sensitivity | 1D specificity |
|---|---|---|---|
| Calcium plaque | 93 | 80 | 94 |
| Fibrous plaque | 90 | 63 | 92 |
| Lipidic plaque | 90 | 79 | 91 |

Cross-Section Image Generation

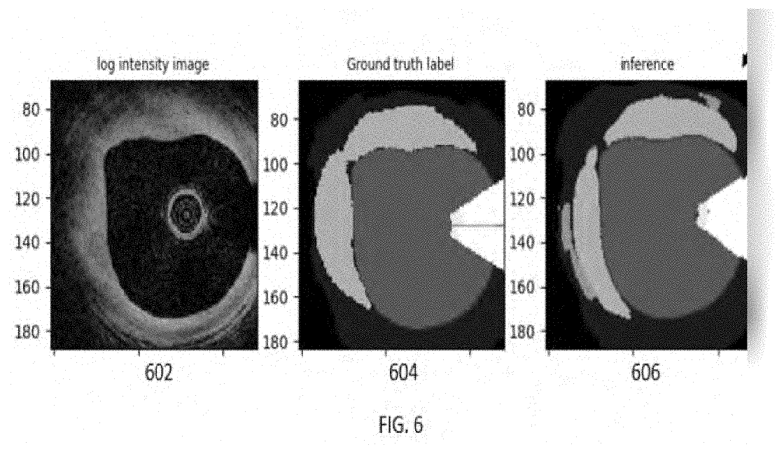

FIG. 6 presents exemplary IVOCT cross-sectional image outputs 600 by the frame-based cross-sectional display 110 in accordance with FIG. 1A. Following further processing of the data by frame-based post processing module 108, as part of the image generation module 408 as presented in FIG. 4, the cross-sectional image outputs 600 were generated. The first output 602 presents a representative IVOCT cross-section in log-scale. The ground truth labels 604 of the IVOCT cross-section and the network inference 606 of the IVOCT cross-section were also generated. The x-axis line in the ground labels 604 is determined to be artifact of coordinate conversion.

Enface Image Generation

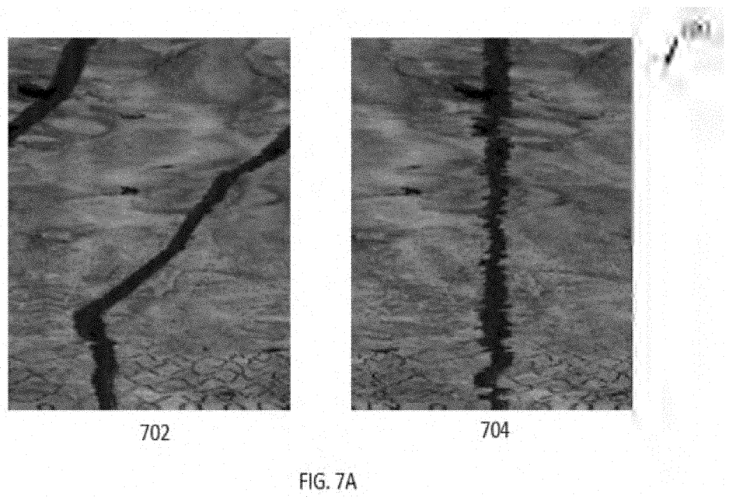

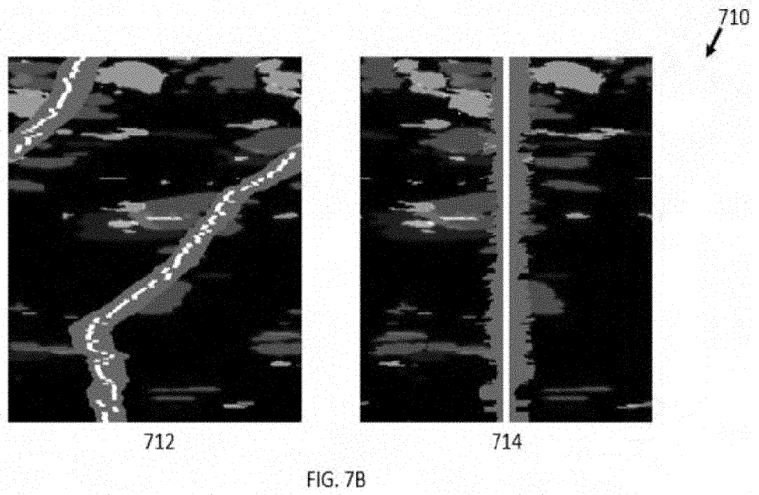

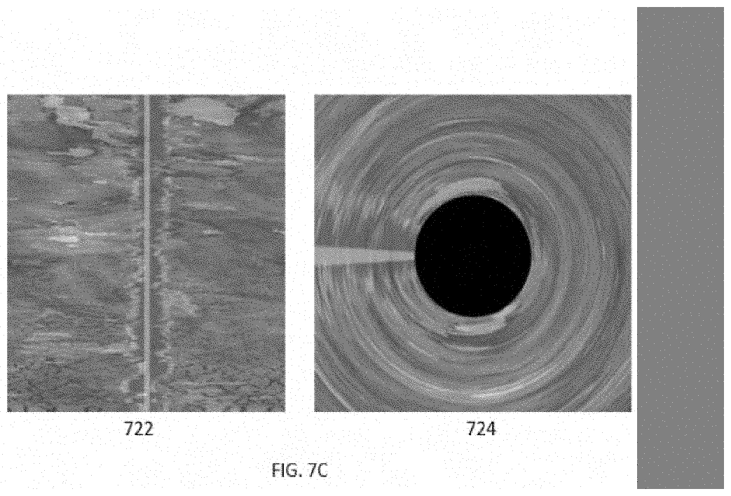

FIGS. 7A-7C present exemplary raw Enface image outputs 700, plaque overlay image outputs 710, and mixed Enface image outputs 720 by the Enface display 116 in accordance with FIG. 1A. Following further processing of the data by scan-based post processing module 114, as part of the image generation module 408 as presented in FIG. 4, the raw Enface image outputs 00, plaque overlay image outputs 710, and mixed Enface image outputs 720 were generated. The Enface image display 116 is commonly used in OCT to summarize the contents of a 3D volume of data in a concise 2D image, projecting the data in the axial direction. In IVOCT, due to the presence of the guide-wire and other non-tissue signal in the lumen, it is helpful to segment out the lumen and guide-wire before projecting the data in the axial direction. The projection of the data in the axial direction can be interpreted as a number of processes which include the maximum value in the axial direction, the minimum value in the axial direction, the mean value in the axial direction, and other custom processing steps in the axial direction

The scan-based feature segmentation module 112, the scan-based post processing module 114 as presented in FIG. 1A were designed with to color the Enface image using a 3-color RGB value that corresponds to the presence or absence of a type of plaque at a given angle and pull-back position, with red denoting if calcium plaque is present, green denoting if fibrous plaque is present, blue denoting if lipidic plaque is present, and a combination of colors denoting if multiple plaques are present at different depths of an A-line. The color values are binarized to reduce the number of possible color combinations in the Enface image. This binarization is achieved through a combination of local smoothing and thresholding. The thresholds for each of the three plaque classes are chosen to be higher than the threshold used for the frame-based post-processing. This is done to ensure better specificity of the Enface view at a scan-level, since we are naturally leaning towards higher sensitivity when projecting along the axial direction. Currently, these thresholds are optimized for visual quality of the Enface view, as assessed qualitatively on a handful of validation scans. In addition to the color overlays, a gray-scale Enface background image is generated for Enface images, which is an axial projection of the OCT data, resembling what is called volume ray casting in 3D image rendering. Finally, to reduce image artifacts associated with rotation of the optical probe during acquisition, the Enface image lines are aligned, so that the guide-wire is located at a consistent angle to provide a spatially more consistent view of the volume.

FIG. 7A presents exemplary raw Enface image outputs 700 with a gray scale Enface background 702 and an equivalent aligned version 704 of the gray scale Enface background 702. FIG. 7B presents the plaque overlay Enface image outputs 710 with an Enface plaque overlay image 712 with colors denoting the presence of plaques and equivalent aligned version 714. FIG. 7C presents mixed Enface images 720 with a transparency-mixed Enface image 722 of the aligned Enface images 704 and 714. A 3D representation image 724 of transparency-mixed Enface image 722 is also presented in FIG. 7C.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 724 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method for detecting plaque and vessel constriction by processing intracoronary optical coherence tomography (IVOCT) pullback data performed by software executed on a computer, the method comprising:
inputting IVOCT pullback data from an imaging device;
performing full semantic segmentation of the images from the IVOCT pullback data with a frame-based segmentation module;
generating a cross-sectional frame-based image of the images from the segmented IVOCT pullback data with a cross-sectional display; and
determining the presence of plaque and vessel constriction by analyzing the cross-sectional frame-based images.

2. The method according to claim 1, further comprising a frame-based pre-processing module for enhancement, normalization, and resizing of the IVOCT pullback data from the imaging device.

3. The method according to claim 1, further comprising a frame-based post processing module to analyze parameters of the segmented IVOCT pullback data from the frame-based segmentation module.

4. The method according to claim 3, where the parameters are selected from a group including categorical cross-entropy loss, pixel-level accuracy, sensitivity, specificity, optimal threshold, maximum threshold, plaque confusion matrix, 2D Dice overlap coefficients, 1D Dice overlap coefficients, and 1D angular ground truth.

5. The method according to claim 1, further including a scan-based feature segmentation module to process the segmented IVOCT pullback data from the frame-based segmentation module.

6. The method according to claim 5, further generating a Enface scan-based image from the scan-based feature segmented IVOCT pullback data from the scan-based feature segmentation module with a Enface display.

7. A system for detecting plaque and vessel constriction by processing intracoronary optical coherence tomography (IVOCT) pullback data performed by software executed on a computer, the system comprising:
an IVOCT device for acquiring IVOCT pullback data from a patient;
a computer for processing the IVOCT pullback data with a method for detecting plaque and vessel constriction, the method comprising:
inputting IVOCT pullback data from an imaging device;
performing full semantic segmentation of the images from the IVOCT pullback data with a frame-based segmentation module;
generating a cross-sectional frame-based image of the images from the segmented IVOCT pullback data with a cross-sectional display; and
determining the presence of plaque and vessel constriction by analyzing the cross-sectional frame-based images; and
a display screen to display the IVOCT pullback data and the cross-sectional frame-based generated by the method.
